(19) United States
(12) Patent Application Publication
(10) Pub. No.: US 2026/0090744 A1
(43) Pub. Date: Apr. 2, 2026

(54) PULSE OXIMETER

(71) Applicant: SEIKO EPSON CORPORATION, Tokyo (JP)

(72) Inventor: Akira IKEDA, CHINO-SHI (JP)

(73) Assignee: SEIKO EPSON CORPORATION, Tokyo (JP)

(21) Appl. No.: 19/339,336

(22) Filed: Sep. 25, 2025

(30) Foreign Application Priority Data
Sep. 27, 2024 (JP) 2024-168887

(51) Int. Cl. A61B 5/1455; A61B 5/00

(52) U.S. Cl. CPC A61B 5/14552; A61B 5/7221; A61B 5/742; A61B 5/746

(57) ABSTRACT

A pulse oximeter includes a saturation measurement unit that measures a blood oxygen saturation of a subject based on detection signals from a red light sensor and an infrared light sensor to obtain a measurement data group, an abnormal value detection unit that detects an abnormal value appearing in the measurement data group, a first data processing unit that generates a first data group by removing an abnormal value interval including the abnormal value and an adjacent time range having a predetermined length, a second data processing unit that generates a second data group by removing a low reliability interval where a reliability indicator calculated for the first data group is lower than a predetermined threshold, and a third data processing unit that performs data interpolation processing on the second data group to generate a third data group having a predetermined time length.

The present application is based on, and claims priority from JP Application Serial Number 2024-168887, filed Sep. 27, 2024, the disclosure of which is hereby incorporated by reference herein in its entirety.

### BACKGROUND

### 1. Technical Field

The present disclosure relates to a pulse oximeter.

### 2. Related Art

A pulse oximeter measures an arterial blood oxygen saturation (SpO2) by irradiating a finger with red light and near infrared light sources and detecting light transmitted through the finger (see JP-A-2004-337605). When an oxygen desaturation index, which is an indicator of sleep apnea syndrome, is tested, the arterial blood oxygen saturation of a subject during sleep is measured by the pulse oximeter.

JP-A-2004-337605 is an example of the related art.

When the arterial blood oxygen saturation of the subject during sleep is measured, when a body of the subject moves during the measurement, in the pulse oximeter, an intensity variation of reflected light of light in skin due to a body motion is mixed as a disturbance signal (body motion artifact). When such a body motion artifact occurs, a pulsatile signal to be detected by the pulse oximeter is masked, and as a result, there is a problem that measurement accuracy of the arterial blood oxygen saturation decreases.

To address the problem of the decrease in the measurement accuracy of the arterial blood oxygen saturation measured by the pulse oximeter described above, processing of removing an abnormal value due to the body motion artifact or the like from measurement data is performed.

However, analysis on an oxygen desaturation index, which is a test indicator of sleep apnea syndrome, requires data over a sufficiently long period, and when abnormal values are frequently removed from the measurement data, the remaining measurement data becomes discrete, and there is a problem that continuous data having a sufficient length for obtaining the oxygen desaturation index cannot be obtained.

### SUMMARY

A pulse oximeter according to a first aspect of the disclosure includes: a red light sensor configured to irradiate a subject with red light and detect the red light transmitted through or reflected from the subject; an infrared light sensor configured to irradiate the subject with infrared light and detect the infrared light transmitted through or reflected from the subject; and a control device configured to process detection signals from the red light sensor and the infrared light sensor, in which the control device includes a saturation measurement unit that measures a blood oxygen saturation of the subject based on the detection signals and records the blood oxygen saturation as a measurement data group, an abnormal value detection unit that detects an abnormal value appearing in the measurement data group, a first data processing unit that calculates an abnormal value interval including an adjacent time range having a predetermined length temporally adjacent to the abnormal value detected by the abnormal value detection unit, and generates a first data group by removing data in the abnormal value interval from the measurement data group, a second data processing unit that calculates a reliability indicator through convolution for the first data group and generates a second data group by removing, from the first data group, data in a low reliability interval where the reliability indicator is lower than a predetermined threshold, and a third data processing unit that performs, on the second data group, data interpolation processing for the removed abnormal value interval and the removed low reliability interval to generate a third data group having a predetermined time length.

A pulse oximeter according to a second aspect of the disclosure includes: a red light sensor configured to irradiate a subject with red light and detect the red light transmitted through or reflected from the subject; an infrared r configured to irradiate the subject with infrared light and detect the infrared light transmitted through or reflected from the subject; a control device configured to process detection signals from the red light sensor and the infrared light sensor; and a display unit configured to display information obtained by the control device, in which the control device generates a measurement data group by measuring a blood oxygen saturation of the subject based on the detection signals, removes an abnormal value from the measurement data group to obtain an oxygen saturation data group, calculates a reliability indicator for the oxygen saturation data group, and displays the oxygen saturation data group and the reliability indicator on the display unit on the same time axis, and performs, on a range where the reliability indicator exceeds a predetermined threshold in the oxygen saturation data group, data interpolation processing for a data missing portion removed as the abnormal value from the measurement data group, displays the threshold on the display unit, and further displays the portion subjected to the data interpolation processing in a display format different from that of the oxygen saturation data group.

A pulse oximeter according to a third aspect of the disclosure includes: a red light sensor configured to irradiate a subject with red light and detect the red light transmitted through or reflected from the subject; an infrared light sensor configured to irradiate the subject with infrared light and detect the infrared light transmitted through or reflected m the subject; a control device configured to process detection signals from the red light sensor and the infrared light sensor; and a display unit configured to display information obtained by the control device, in which the control device measures a blood oxygen saturation of the subject based on the detection signals to generate a measurement data group, calculates a reliability indicator for the measurement data group, and displays the measurement data group, the reliability indicator, and a time on the display unit, and displays a range exceeding a predetermined threshold and a range below the threshold in the measurement data group in different display formats.

### BRIEF DESCRIPTION OF THE DRAWINGS

FIG. 1 is a schematic diagram showing a pulse oximeter according to an embodiment of the disclosure.

FIG. 2 is a schematic view showing a use state of the pulse oximeter according to the embodiment.

FIG. 3 is a block diagram showing a control device of the pulse oximeter according to the embodiment.

FIG. 4 is a graph showing a measurement data group and an abnormal value in the embodiment.

FIG. 5 is a graph showing an abnormal value interval in the embodiment.

FIG. 6 is a partial enlarged graph of the abnormal value interval in the embodiment.

FIG. 7 is a graph showing a first data group in the embodiment.

FIG. 8 is a graph showing a reliability indicator in the embodiment.

FIG. 9 is a graph showing a second data group in the embodiment.

FIG. 10 is a graph showing a third data group in the embodiment.

FIG. 11 is a schematic diagram showing a display screen in the embodiment.

FIG. 12 is a flowchart showing processing in the embodiment.

FIG. 13 is a schematic diagram showing another embodiment of the disclosure.

### DESCRIPTION OF EMBODIMENTS

An embodiment of the disclosure will be described below.

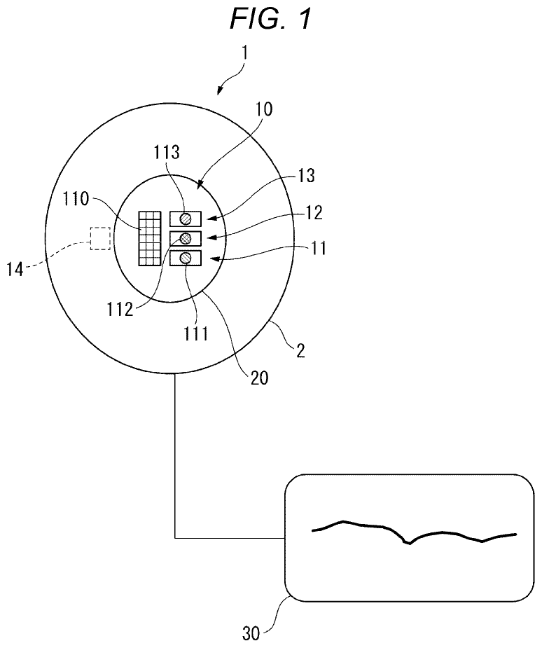

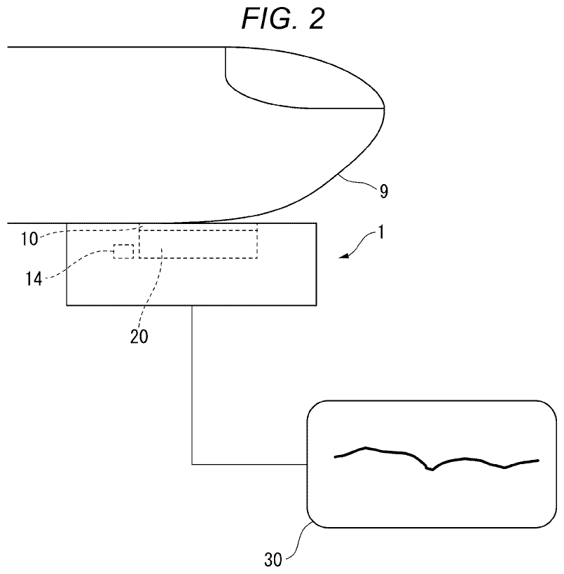

In FIGS. 1 and 2, a pulse oximeter 1 includes a sensor unit 10 and a control device 20. The sensor unit 10 and the control device 20 are accommodated in a case 2, and a display unit 30 is connected to the control device 20.

The sensor unit 10 is disposed on a surface of the case 2 and is held in close contact with a subject 9 such as a finger of a user during measurement.

A green light source 111, a red light source 112, an infrared light source 113, and an optical detector 110 are disposed in the sensor unit 10.

The green light source 111, the red light source 112, and the infrared light source 113 are, for example, light emitting diodes (LEDs) or laser diodes. It is desirable that an emission wavelength of the green light source 111 is 500 nm to 600 nm, an emission wavelength of the red light source 112 is 600 nm to 800 nm, and an emission wavelength of the infrared light source 113 is 800 nm to 1,000 nm.

The optical detector 110 is, for example, a silicon photodiode. Surfaces of the green light source 111, the red light source 112, the infrared light source 113, and the optical detector 110 of the sensor unit 10 are covered with a cover (not shown) made of a transparent resin-molded plate, such as acrylic or polycarbonate.

In the sensor unit 10, the green light source 111, the red light source 112, and the infrared light source 113 sequentially emit light under control of the control device 20, and light beams from the respective light sources are reflected by subcutaneous tissue of the subject 9 and returned to the optical detector 110. The control device 20 acquires detection signals from the optical detector 110 as a detection signal of green light, a detection signal of red light, and a detection signal of infrared light from a light emission timing of each light emitting element. The green light source 111 and the optical detector 110 constitute a green light sensor 11, the red light source 112 and the optical detector 110 constitute a red light sensor 12, and the infrared light source 113 and the optical detector 110 constitute an infrared light sensor 13.

The sensor unit 10 further includes an acceleration sensor 14 provided in the case 2.

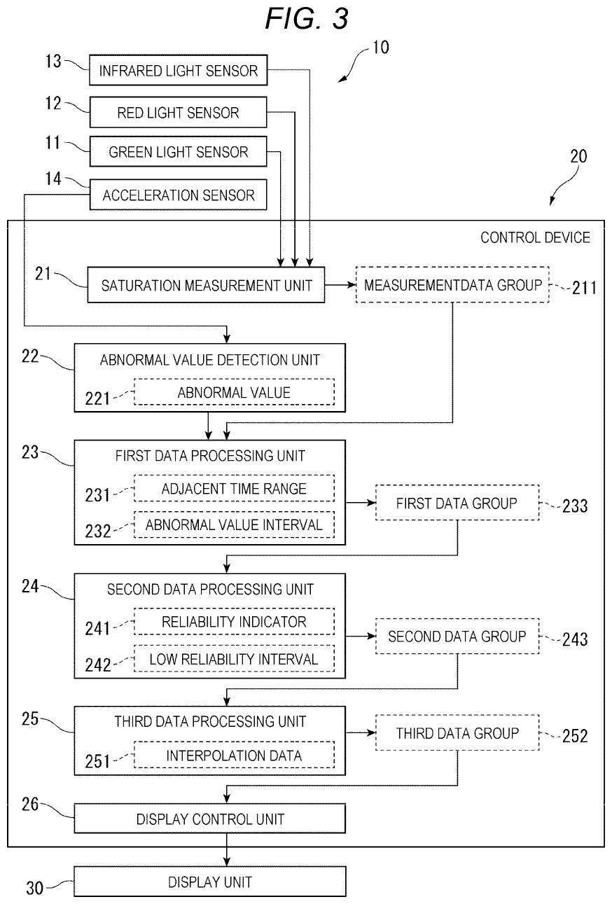

The control device 20 is implemented by a small computer system and includes a memory that stores various data and a processor that implements a desired function (see FIG. 3) by executing a program stored in the memory.

In the case 2, a battery serving as a power supply of the sensor unit 10 and the control device 20 is stored, and an input and output terminal or the like of the control device 20 is provided (not shown).

The display unit 30 is connected to the control device 20 by a wired or wireless signal unit, and can display a measurement result or the like from the control device 20. As the display unit 30, for example, in addition to a general-purpose image display panel, a portable information terminal such as a so-called smartphone can be used.

In FIG. 3, the control device 20 functions as a saturation measurement unit 21, an abnormal value detection unit 22, a first data processing unit 23, a second data processing unit 24, a third data processing unit 25, and a display control unit 26 by the processor executing a program.

The saturation measurement unit 21 controls the green light sensor 11, the red light sensor 12, and the infrared light sensor 13 of the sensor unit 10 connected to the control device 20, and acquires (receives) a detection signal.

The saturation measurement unit 21 measures a blood oxygen saturation of the subject 9 based on detection signals from the red light sensor 12 and the infrared light sensor 13, and records the blood oxygen saturation in the memory as a measurement data group 211. The detection signals from the red light sensor 12 and the infrared light sensor 13 are photoplethysmographic signals based on red light and infrared light transmitted through the subcutaneous tissue of the subject 9, and the saturation measurement unit 21 measures the oxygen saturation based on red light and infrared light pulse wave signals and records the oxygen saturation in the memory as the measurement data group 211.

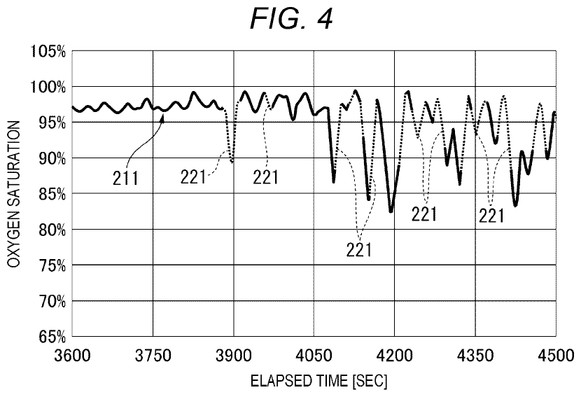

The abnormal value detection unit 22 detects an abnormal value 221 (see FIG. 4) appearing in the measurement data group 211. Therefore, the abnormal value detection unit 22 processes a detection signal from the acceleration sensor 14, detects a body motion artifact such as a posture change of the subject 9, and determines, in response to a body motion artifact larger than a predetermined reference value, a state in which an anomaly occurs in oxygen saturation measurement by the saturation measurement unit 21. Then, the measurement data group 211 in a period in which it is determined that the anomaly occurs is detected as the abnormal value 221.

FIG. 4 shows an example of the measurement data group 211 acquired by the saturation measurement unit 21. In the measurement data group 211, a plurality of intervals may be detected as the abnormal value 221 by the abnormal value detection unit 22.

The first data processing unit 23 calculates an abnormal value interval 232 including an adjacent time range 231 having a predetermined length temporally adjacent to the abnormal value 221 detected by the abnormal value detection unit 22, and generates a first data group 233 by removing data in the abnormal value interval 232 from the measurement data group 211.

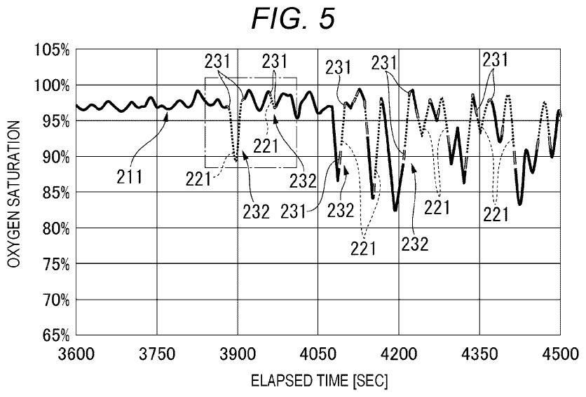

In FIG. 5, for the measurement data group 211 where the abnormal value 221 is detected, the first data processing unit 23 delineates the adjacent time range 231 having the predetermined length in the measurement data group 211 adjacent to each of both ends (temporal start and end) of each abnormal value 221, and combines a pair of adjacent time ranges 231 on both sides with the abnormal value 221 therebetween to form the abnormal value interval 232.

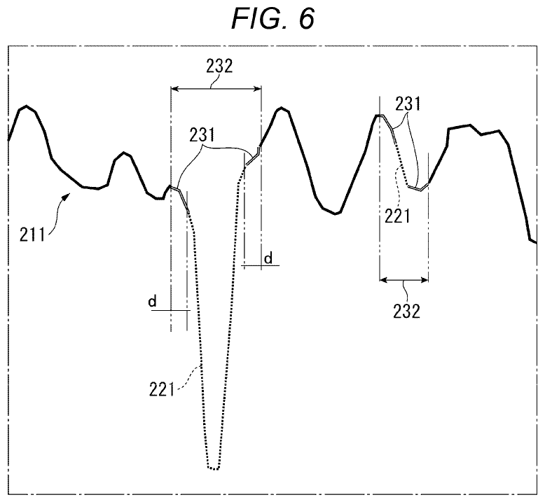

FIG. 6 is an enlarged view of a portion indicated by a one-dot chain line in FIG. 5.

In FIG. 6, the abnormal value interval 232 includes the abnormal value 221 and the adjacent time ranges 231 on both sides thereof. A length d of each adjacent time range 231 is a preset time, for example, 2 seconds. The adjacent time ranges 231 are not the abnormal value 221 determined to be abnormal by the abnormal value detection unit 22, and the abnormal value interval 232 is extended temporally before and after the original abnormal value 221 by connecting the adjacent time ranges 231.

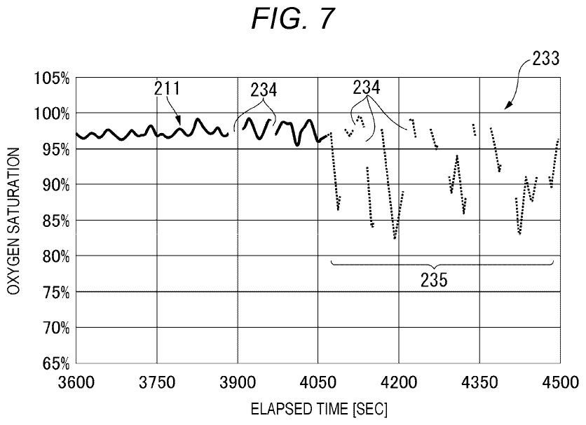

As described above, the first data processing unit 23 generates the first data group 233 (see FIG. 7) by removing the data in the abnormal value interval 232 from the measurement data group 211.

In FIG. 7, in the first data group 233, the data missing portion 234 is formed after the abnormal value interval 232 is removed from the measurement data group 211. Such a data missing portion 234 occurs at a high frequency in a partial region 235 in the first data group 233.

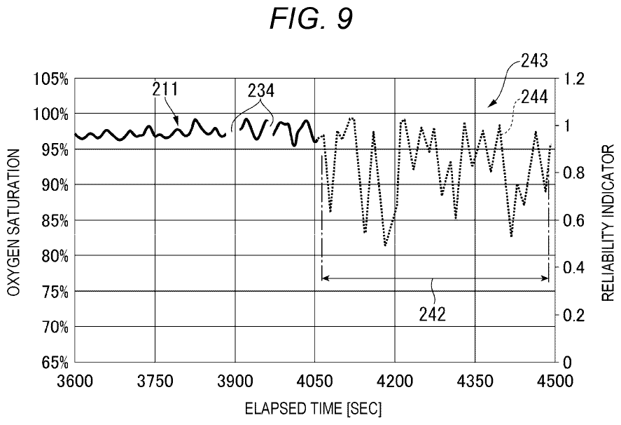

The second data processing unit 24 calculates a reliability indicator 241 through convolution for the first data group 233 generated by the first data processing unit 23, and generates a second data group 243 (see FIG. 9) by removing, from the first data group 233, data in a low reliability interval 242 where the reliability indicator 241 is lower than a predetermined threshold.

As the reliability indicator 241, a ratio (0 to 1) of data remaining after removing the abnormal value interval 232 from the measurement data group 211 to the data in the measurement data group 211 can be used. When the reliability indicator 241 at any time in the measurement data group 211 is calculated, a convolution operation can be used.

For example, measurement data of the oxygen saturation at a time t is f(t). The measurement data f(t) is non-negative data, and the abnormal value is 0. A convolution function g(n) for determining an interval that is abnormal data for the measurement data f(t) is as follows.

$$g ( n ) = \frac{1}{n} \{ \begin{matrix}1 \cdot f ( j ) > 0 ) \\ 0 \cdot ( f ( j ) = 0 )\end{matrix}$$

A convolution result of the measurement data f(t) by a convolution function g(t) is defined as a reliability indicator T(t)=f(t)·g(t). According to the convolution function g(t), the reliability indicator T(t) represents a sufficiency rate of the measurement data in a convolution interval, that is, how much data remains after the abnormal data is removed from the measurement data. A possible range of the reliability indicator T(t) is 0 to 1. The predetermined threshold used for the determination of the reliability indicator 241 is appropriately set based on a distribution of the first data group 233 and the data missing portion 234, specifically, to include the region 235 where the data missing portion 234 occurs at a high frequency in the first data group 233, and for example, the threshold for the reliability indicator 241 is 0.7.

In FIG. 7 described above, in the first data group 233, the data missing portion 234 occurs at a high frequency in the partial region 235.

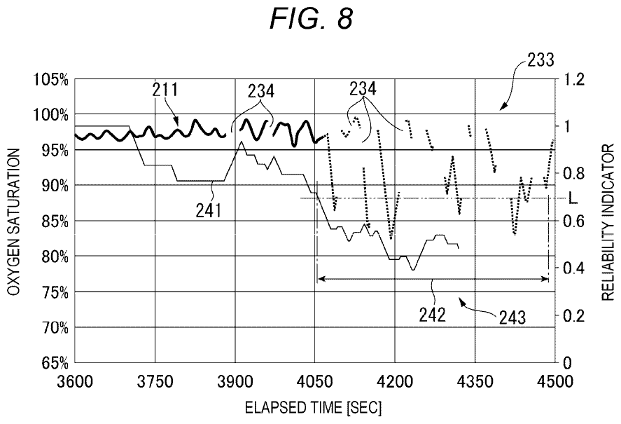

In FIG. 8, when the first data group 233 and the reliability indicator 241 are displayed on the same time axis, a threshold L=0.7 is set. The low reliability interval 242 is specified as a region where the reliability indicator 241 is equal to or less than the threshold L=0.7, and this region corresponds to the region 235 (see FIG. 7) where the data missing portion 234 described above occurs at a high frequency.

In FIG. 9, in the second data group 243, a data missing portion 244 is formed after the low reliability interval 242 is removed from the first data group 233. In a portion other than the low reliability interval 242, the data missing portion 234 after the abnormal value interval 232 is removed from the measurement data group 211 remains.

A time step for processing of determining the low reliability interval 242 from the reliability indicator 241 is preferably the same as the number of points in the convolution operation. When the number of convolution points is 180 points (180 seconds) with respect to data with a sampling interval of one second, the time step for evaluating the reliability indicator 241 may also be 180 seconds.

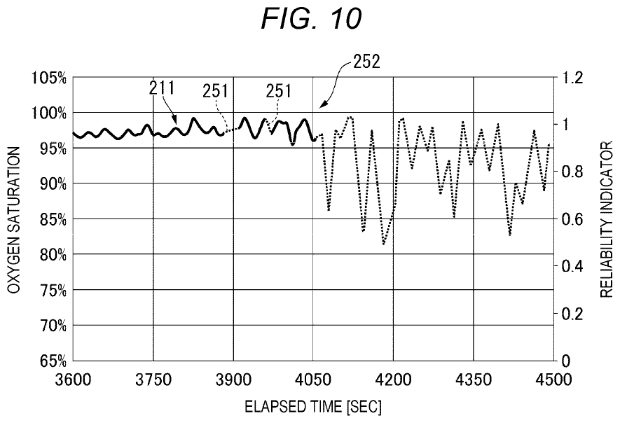

The third data processing unit 25 performs, on the second data group 243 generated by the second data processing unit 24, data interpolation processing for the abnormal value interval 232 (data missing portion 234) and the low reliability interval 242 (data missing portion 244) to add interpolation data 251 and generate a third data group 252 (see FIG. 10) having a predetermined time length.

Any data interpolation method such as linear interpolation or spline interpolation can be used for the data interpolation processing. When a duration of the data missing portions 234 and 244 is longer than a predetermined reference value, the data interpolation may be stopped and excluded from the third data group 252.

As a duration of the third data group 252, it is desirable to secure 180 seconds required for analysis on an oxygen desaturation index, which is a test indicator of sleep apnea syndrome.

The display control unit 26 displays various data obtained by the control device 20 on the display unit 30.

As display contents, in addition to using the third data group 252 obtained by the third data processing unit 25 and the reliability indicator 241 obtained by the second data processing unit 24, the measurement data group 211 that is a basis for the measurement, or the first data group 233 or the second data group 243, which is intermediate processed data, may be displayed, and an alert display or the like based on these may be displayed.

Further, the third data group 252 may be used to calculate an interval where a blood oxygen level decreases over a predetermined time or the number of times the blood oxygen level decreases, and a calculation result may be displayed on the display unit 30.

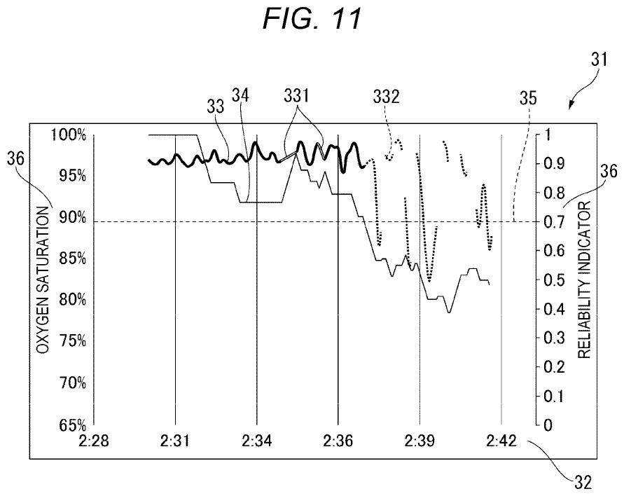

FIG. 11 shows an example of a display screen 31 displayed on the display unit 30.

On the display screen 31, an oxygen saturation graph 33 indicating the third data group 252 generated by the third data processing unit 25, a reliability indicator graph 34 indicating the reliability indicator 241, and a threshold display line 35 indicating the threshold L are displayed along a time axis 32 displayed at a lower part.

Among these, the oxygen saturation graph 33 and the reliability indicator graph 34 are displayed in a display format that is easily distinguished from vertical axis displays 36 on both sides, for example, in a display format having different saturation, brightness, transparency, or line types, or a combination thereof. The threshold display line 35 is displayed in a display format according to the vertical axis displays 36. The threshold display line 35 is not limited to a horizontal straight line added to the display of the reliability indicator graph 34, and may be displayed by another mark or shape.

Further, in a part of the oxygen saturation graph 33, an interpolation data display 331 indicating the interpolation data 251 for the data missing portion 234 is displayed, and a low reliability interval display 332 for the low reliability interval 242 (a range below the threshold L) is provided. The interpolation data display 331 and the low reliability interval display 332 are each displayed in a display format that is easily distinguished from other portions (a range exceeding the threshold L) of the oxygen saturation graph 33, for example, in a display format having different saturation, brightness, transparency, or line types, or a combination thereof. For the low reliability interval display 332, the data missing portion 234 may remain without being subjected to data interpolation, and may be displayed in gray or the like to be less distinguishable than the other portions of the oxygen saturation graph 33.

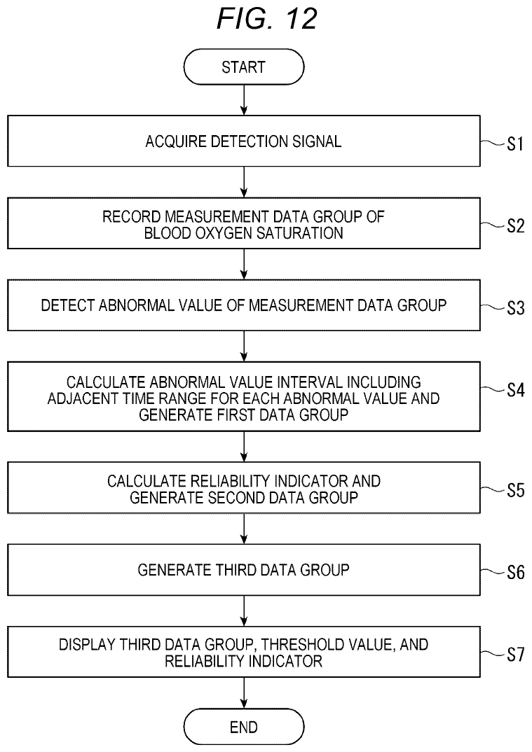

FIG. 12 shows an overview of processing executed by the pulse oximeter 1 of the embodiment.

In the pulse oximeter 1, when the processor of the control device 20 executes a program, the following processing is executed by each unit of the sensor unit 10 and the control device 20.

In processing S1, the sensor unit 10 irradiates the subject 9 with red light and infrared light and detects the red light and the infrared light transmitted through or reflected from the subject 9.

In processing S2, the saturation measurement unit 21 measures the blood oxygen saturation of the subject 9 based on the detection signals of the red light and the infrared light, and records the blood oxygen saturation as the measurement data group 211 (see FIG. 4).

In processing S3, the abnormal value detection unit 22 detects the abnormal value 221 appearing in the measurement data group 211 (see FIG. 4).

In processing S4, the first data processing unit 23 calculates the abnormal value interval 232 including the adjacent time range 231 having a predetermined length temporally adjacent to the abnormal value 221 and generates the first data group 233 by removing the data in the abnormal value interval 232 from the measurement data group 211 (see FIGS. 5 and 6).

In processing S5, the second data processing unit 24 calculates the reliability indicator 241 through convolution for the first data group 233 and generates the second data group 243 by removing, from the first data group 233, the data in the low reliability interval 242 where the reliability indicator 241 is lower than the predetermined threshold L (see FIG. 9).

In processing S6, the third data processing unit 25 performs, on the second data group 243, data interpolation processing for the removed abnormal value interval 232 and the removed low reliability interval 242 to generate the third data group 252 having a predetermined time length (see FIG. 10).

In processing S6, the display control unit 26 displays the third data group 252, the threshold L, and the reliability indicator 241 on the display unit 30 (see FIG. 11).

### Functions and Effects of Embodiment

The pulse oximeter 1 of the embodiment includes the red light sensor 12 that irradiates the subject 9 with red light and detects the red light transmitted through or reflected from the subject 9, the infrared light sensor 13 that irradiates the subject 9 with infrared light and detects the infrared light transmitted through or reflected from the subject 9, and the control device 20 that processes the detection signals from the red light sensor 12 and the infrared light sensor 13.

The control device 20 includes the saturation measurement unit 21 that measures the blood oxygen saturation of the subject 9 based on the detection signals from the red light sensor 12 and the infrared light sensor 13 and records the blood oxygen saturation as the measurement data group 211, the abnormal value detection unit 22 that detects the abnormal value 221 appearing in the measurement data group 211, the first data processing unit 23 that calculates the abnormal value interval 232 including the adjacent time range 231 having the predetermined length temporally adjacent to the abnormal value 221 detected by the abnormal value detection unit 22 and generates the first data group 233 by removing the data in the abnormal value interval 232 from the measurement data group 211, the second data processing unit 24 that calculates the reliability indicator 241 through convolution for the first data group 233 and generates the second data group 243 by removing the data in the low reliability interval 242 where the reliability indicator 241 is lower than the predetermined threshold L from the first data group 233, and the third data processing unit 25 that performs data interpolation processing on the second data group 243 for the removed abnormal value interval 232 and the removed low reliability interval 242 to generate the third data group 252 having the predetermined time length.

In such a pulse oximeter 1, the measurement data group 211 of the blood oxygen saturation of the subject 9 is obtained by the red light sensor 12, the infrared light sensor 13, and the saturation measurement unit 21 of the control device 20.

By detecting the abnormal value 221 by the abnormal value detection unit 22 with respect to the measurement data group 211, a measurement error caused by a body motion of the subject 9 can be removed to improve measurement accuracy.

When removing the abnormal value 221 from the measurement data group 211, the first data processing unit 23 uses the abnormal value interval 232 including the adjacent time range 231 having the predetermined length temporally adjacent to the abnormal value 221, and thus it is possible to also remove false values generated before and after data detected as the abnormal value 221 to further improve the measurement accuracy.

Further, when removing the abnormal value 221 from the measurement data group 211, the second data processing unit 24 calculates the reliability indicator 241 through convolution for the first data group 233 and removes the low reliability interval 242 where the reliability indicator 241 is lower than the predetermined threshold L from the measurement data group 211 together with the abnormal value interval 232, and thus an interval having less valid data can be excluded from the measurement to further improve the measurement accuracy.

By the above processing, measurement accuracy of an arterial blood oxygen saturation is improved, and by performing the data interpolation processing using the third data processing unit 25, continuous data having a length sufficient to obtain the oxygen desaturation index is obtained.

The pulse oximeter 1 of the embodiment further includes the display unit 30 that displays information obtained by the control device 20 to allow the user who is the subject 9, an examiner, or the like to recognize a test result.

The display unit 30 can display the alert display based on the measurement result in addition to the measurement result of the blood oxygen saturation by the control device 20. These display contents are displayed in different display formats based on the reliability indicator 241 to improve distinguishability of the measurement result for the user. Further, the third data group 252 can be used to calculate the interval where the blood oxygen level decreases over the predetermined time or the number of times the blood oxygen level decreases, and the calculation result may also be displayed on the display unit 30.

The pulse oximeter 1 of the embodiment includes the red light sensor 12 that irradiates the subject 9 with red light and detects the red light transmitted through or reflected from the subject 9, the infrared light sensor 13 that irradiates the subject 9 with infrared light and detects the infrared light transmitted through or reflected from the subject 9, the control device 20 that processes detection signals from the red light sensor 12 and the infrared light sensor 13, and the display unit 30 that displays information obtained by the control device 20. The control device 20 measures the blood oxygen saturation of the subject 9 based on the detection signals to generate the measurement data group 211, removes the abnormal value 221 from the measurement data group 211 to obtain the oxygen saturation data group (first data group 233), calculates the reliability indicator 241 for the oxygen saturation data group, displays the oxygen saturation data group (233) and the reliability indicator 241 on the display unit 30 on the same time axis, and performs, on the range where the reliability indicator 241 exceeds the predetermined threshold L in the oxygen saturation data group, the data interpolation processing for the data missing portion 234 removed as the abnormal value 221 from the measurement data group 211, displays the threshold L on the display unit 30, and further displays the portion (data missing portion 234) subjected to the data interpolation processing in a display format different from that of the oxygen saturation data group (233).

In such a pulse oximeter 1, the red light sensor, the infrared light sensor, and the control device 20 can measure the blood oxygen saturation of the subject and display the measurement result on the display unit.

In the control device 20, by removing the abnormal value 221 from the measurement data group 211 obtained from the red light sensor 12 and the infrared light sensor 13 to obtain the oxygen saturation data group (first data group 233), a measurement error caused by a body motion of the subject can be removed to improve measurement accuracy. Further, by displaying the oxygen saturation data group together with the reliability indicator 241 thereof on the same time axis, the user can recognize the reliability of the measurement result. For the oxygen saturation data group, by performing the data interpolation processing for the data missing portion 234 removed from the measurement data group 211, the continuous data (third data group 252) having a length sufficient to obtain the oxygen desaturation index is obtained. By displaying the portion (data missing portion 234) subjected to the data interpolation processing together with the threshold L of the reliability indicator 241 in different display formats on the display unit 30, the reliability of the measurement result can be recognized by the user also in this respect.

### Other Embodiments

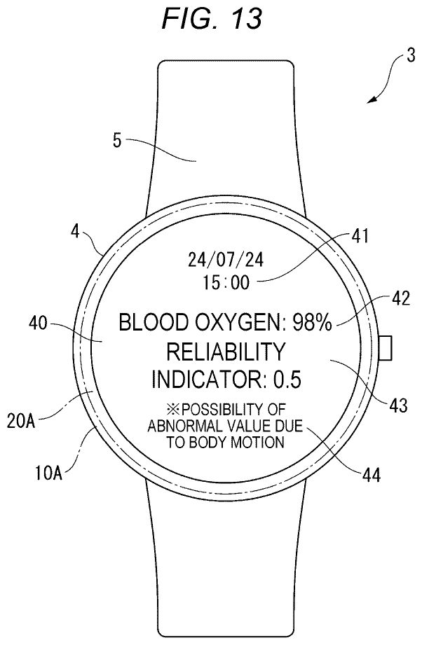

FIG. 13 shows another embodiment of the disclosure.

In FIG. 13, a pulse oximeter 3 has a flat disk-shaped case 4 and a belt 5, and can be worn on a wrist of the user.

A display unit 40 is formed at a surface of the case 4, and a current time display 41, an oxygen saturation display 42, a reliability indicator display 43, and an alert display 44 are displayed on the display unit 40. A control device 20A is stored inside the case 4, and a sensor unit 10A is formed at a back side of the case 4.

The sensor unit 10A includes the red light sensor 12 and the infrared light sensor 13 similarly to the sensor unit 10 of the embodiment described above. The red light sensor 12 and the infrared light sensor 13 are disposed to be in close contact with a surface of the wrist of the user when the pulse oximeter 3 is attached to the wrist of the user with the belt 5, and the surface of the wrist serves as the subject 9.

The control device 20A is implemented similarly to the control device 20 described above, measures the blood oxygen saturation of the subject 9 based on the detection signals from the red light sensor 12 and the infrared light sensor 13 to generate the measurement data group 211, and calculates the reliability indicator 241 for the measurement data group 211.

The control device 20A displays the oxygen saturation data at the current time or any time in the past of the measurement data group 211 on the oxygen saturation display 42 and displays the reliability indicator 241 of the same time on the reliability indicator display 43 based on an operation of the user.

When displaying the oxygen saturation display 42 and the reliability indicator display 43, the control device 20A compares the reliability indicator 241 at the time of display with the predetermined threshold L, and displays a range exceeding the threshold L and a range below the threshold L in different display formats.

Specifically, in a state where the reliability indicator 241 falls below the threshold L, the control device 20A causes the alert display 44 to display a character or a symbol regarding a possibility of an abnormal value due to a body motion, sets the display of the reliability indicator display 43 to a display format different from that in a state where the reliability indicator 241 exceeds the threshold L, and sets the display of the oxygen saturation display 42 to a display format different from that in the state where the reliability indicator 241 exceeds the threshold L. Differentiation of the display format in the state where the reliability indicator 241 falls below the threshold L may be only one of the above three. As the difference in the display format, for example, a display format different in saturation, brightness, transparency, line type, or a combination thereof can be used.

The pulse oximeter 3 of the embodiment includes the red light sensor 12 that irradiates the subject 9 with red light and detects the red light transmitted through or reflected from the subject 9, the infrared light sensor 13 that irradiates the subject 9 with infrared light and detects the infrared light transmitted through or reflected from the subject 9, the control device 20A that processes detection signals from the red light sensor 12 and the infrared light sensor 13, and the display unit 40 that displays information obtained by the control device 20A. The control device 20A is configured to measure the blood oxygen saturation of the subject 9 based on the detection signals from the red light sensor 12 and the infrared light sensor 13 to generate the measurement data group 211, calculate the reliability indicator 241 for the measurement data group 211, display the measurement data group 211, the reliability indicator 241, and a time on the display unit 40, and display the range exceeding the predetermined threshold L and the range below the threshold L in the measurement data group 211 in different display formats.

In such a pulse oximeter 3, the red light sensor 12, the infrared light sensor 13, and the control device 20A can measure the blood oxygen saturation of the subject 9 and display the measurement result on the display unit 40.

In the control device 20A, the measurement data group 211 obtained from the red light sensor 12 and the infrared light sensor 13 is displayed together with the reliability indicator 241 and the time, and in particular, the range exceeding the predetermined threshold L and the range below the threshold L in the measurement data group 211 are displayed in different display formats to allow the user to recognize the reliability of the measurement result.

### Modifications

The disclosure is not limited to the embodiments described above, and modifications and the like within a range where the object of the disclosure can be obtained are contained in the disclosure.

The pulse oximeter of the disclosure is not limited to the reflective type described in the embodiment, and can also be applied to a transmissive type.

For the determination of the abnormal value 221 in the abnormal value detection unit 22, in addition to detecting the body motion of the subject by a physical detection method such as the acceleration sensor 14 and determining the measurement value of the blood oxygen saturation at the time when the body motion is detected as the abnormal value, other methods such as determining an abnormal numerical value for the data in the measurement data group 211 of the blood oxygen saturation may be used.

In the present specification, the pulse oximeter that measures the blood oxygen saturation has been described, and it is needless to say that the disclosure can be used not only in a device called a pulse oximeter but also in a blood oxygen wellness apparatus that displays a measurement result as a “blood oxygen level”. It should be understood that the pulse oximeter includes the blood oxygen wellness apparatus in this specification.

### Summary of Disclosure

A pulse oximeter according to a first aspect of the disclosure includes: a red light sensor configured to irradiate a subject with red light and detect the red light transmitted through or reflected from the subject; an infrared light sensor configured to irradiate the subject with infrared light and detect the infrared light transmitted through or reflected from the subject; and a control device configured to process detection signals from the red light sensor and the infrared light sensor, in which the control device includes a saturation measurement unit that measures a blood oxygen saturation of the subject based on the detection signals and records the blood oxygen saturation as a measurement data group, an abnormal value detection unit that detects an abnormal value appearing in the measurement data group, a first data processing unit that calculates an abnormal value interval including an adjacent time range having a predetermined length temporally adjacent to the abnormal value detected by the abnormal value detection unit, and generates a first data group by removing data in the abnormal value interval from the measurement data group, a second data processing unit that calculates a reliability indicator through convolution for the first data group and generates a second data group by removing, from the first data group, data in a low reliability interval where the reliability indicator is lower than a predetermined threshold, and a third data processing unit that performs, on the second data group, data interpolation processing for the removed abnormal value interval and the removed low reliability interval to generate a third data group having a predetermined time length.

In such a pulse oximeter, the measurement data group of the blood oxygen saturation of the subject is obtained by the red light sensor, the infrared light sensor, and the saturation measurement unit of the control device.

By detecting an abnormal value by the abnormal value detection unit with respect to the measurement data group, a measurement error caused by a body motion of the subject can be removed to improve measurement accuracy.

When removing the abnormal value from the measurement data group, the first data processing unit uses the abnormal value interval including the adjacent time range having the predetermined length temporally adjacent to the abnormal value, and thus it is possible to also remove false values generated before and after data detected as the abnormal value to further improve the measurement accuracy.

Further, when removing the abnormal value from the measurement data group, the second data processing unit calculates the reliability indicator through convolution for the first data group and removes the low reliability interval where the reliability indicator is lower than the predetermined threshold from the measurement data group together with the abnormal value interval, and thus an interval having less valid data can be excluded from the measurement to further improve the measurement accuracy.

By the above processing, measurement accuracy of an arterial blood oxygen saturation is improved, and by performing the data interpolation processing using the third data processing unit, continuous data having a length sufficient to obtain an oxygen desaturation index is obtained.

The pulse oximeter according to the first aspect of the disclosure may further include a display unit configured to display information obtained by the control device, in which the control device may calculate, using the third data group, an interval where a blood oxygen level decreases over a predetermined time or the number of times the blood oxygen level decreases, and display a calculation result on the display unit.

A pulse oximeter according to a second aspect of the disclosure includes: a red light sensor configured to irradiate a subject with red light and detect the red light transmitted through or reflected from the subject; an infrared light sensor configured to irradiate the subject with infrared light and detect the infrared light transmitted through or reflected from the subject; a control device configured to process detection signals from the red light sensor and the infrared light sensor; and a display unit configured to display information obtained by the control device, in which the control device generates a measurement data group by measuring a blood oxygen saturation of the subject based on the detection signals, removes an abnormal value from the measurement data group to obtain an oxygen saturation data group, calculates a reliability indicator for the oxygen saturation data group, displays the oxygen saturation data group and the reliability indicator on the display unit on the same time axis, and performs, on a range where the reliability indicator exceeds a predetermined threshold in the oxygen saturation data group, data interpolation processing for a data missing portion removed as the abnormal value from the measurement data group, displays the threshold on the display unit, and further displays the portion subjected to the data interpolation processing in a display format different from that of the oxygen saturation data group.

In such a pulse oximeter, the red light sensor, the infrared light sensor, and the control device can measure the blood oxygen saturation of the subject and display the measurement result on the display unit.

In the control device, by removing the abnormal value from the measurement data group obtained from the red light sensor and the infrared light sensor to obtain the oxygen saturation data group, a measurement error caused by a body motion of the subject can be removed to improve measurement accuracy. Further, by displaying the oxygen saturation group together with the reliability indicator thereof on the same time axis, a user can recognize reliability of the measurement result. For the oxygen saturation data group, by performing the data interpolation processing for the data missing portion removed from the measurement data group, continuous data having a length sufficient to obtain an oxygen desaturation index is obtained. By displaying the portion subjected to the data interpolation processing together with the threshold of the reliability indicator in different display formats on the display unit, the reliability of the measurement result can be recognized by the user also in this respect.

In the pulse oximeter according to the second aspect of the disclosure, the control device may calculate an interval where a blood oxygen level decreases over a predetermined time or the number of times the blood oxygen level decreases, and display a calculation result on the display unit.

In the pulse oximeter according to the second aspect of the disclosure, the threshold may be displayed by a mark or a shape added to the display of the reliability indicator.

In the pulse oximeter according to the second aspect of the disclosure, a range exceeding the threshold and a range below the threshold in the measurement data group may be displayed in different display formats.

In the pulse oximeter according to the second aspect of the disclosure, the different display formats between the range exceeding the threshold and the range below the threshold may be any one or a combination of saturation, brightness, and transparency.

In the pulse oximeter according to the second aspect of the disclosure, different display formats between the portion subjected to the data interpolation processing and the original measurement data group may be any one or a combination of saturation, brightness, and transparency.

A pulse oximeter according to a third aspect of the disclosure includes: a red light sensor configured to irradiate a subject with red light and detect the red light transmitted through or reflected from the subject; an infrared light sensor configured to irradiate the subject with infrared light and detect the infrared light transmitted through or reflected from the subject; a control device configured to process detection signals from the red light sensor and the infrared light sensor; and a display unit configured to display information obtained by the control device, in which the control device measures a blood oxygen saturation of the subject based on the detection signals to generate a measurement data group, calculates a reliability indicator for the measurement data group, and displays the measurement data group, the reliability indicator, and a time on the display unit, and displays a range exceeding a predetermined threshold and a range below the threshold in the measurement data group in different display formats.

In such a pulse oximeter, the red light sensor, the infrared light sensor, and the control device can measure the blood oxygen saturation of the subject and display the measurement result on the display unit.

In the control device, the measurement data group obtained from the red light sensor and the infrared light sensor is displayed together with the reliability indicator and the time, and in particular, the range exceeding the predetermined threshold and the range below the threshold in the measurement data group are displayed in different display formats to allow the user to recognize the reliability of the measurement result.

In the pulse oximeter according to the third aspect of the disclosure, in a state where the reliability indicator falls below the threshold, one or a combination of the following may be performed: displaying a character or a symbol regarding a possibility of an abnormal value due to a body motion, displaying the reliability indicator in a display format different from that in a state where the reliability indicator exceeds the threshold, and displaying the measurement data group in a display format different from that in the state where the reliability indicator exceeds the threshold.

What is claimed is:

1. A pulse oximeter comprising:
a red light sensor configured to irradiate a subject with red light and detect the red light transmitted through or reflected from the subject;
an infrared light sensor configured to irradiate the subject with infrared light and detect the infrared light transmitted through or reflected from the subject; and
a control device configured to process detection signals from the red light sensor and the infrared light sensor, wherein
the control device includes
a saturation measurement unit that measures a blood oxygen saturation of the subject based on the detection signals and records the blood oxygen saturation as a measurement data group,
an abnormal value detection unit that detects an abnormal value appearing in the measurement data group,
a first data processing unit that calculates an abnormal value interval including an adjacent time range having a predetermined length temporally adjacent to the abnormal value detected by the abnormal value detection unit, and generates a first data group by removing data in the abnormal value interval from the measurement data group,
a second data processing unit that calculates a reliability indicator through convolution for the first data group and generates a second data group by removing, from the first data group, data in a low reliability interval where the reliability indicator is lower than a predetermined threshold, and
a third data processing unit that performs, on the second data group, data interpolation processing for the removed abnormal value interval and the removed low reliability interval to generate a third data group having a predetermined time length.

2. The pulse oximeter according to claim 1, further comprising:
a display unit configured to display information obtained by the control device, wherein
the control device calculates, using the third data group, an interval where a blood oxygen level decreases over a predetermined time or the number of times the blood oxygen level decreases, and displays a calculation result on the display unit.

3. A pulse oximeter comprising:
a red light sensor configured to irradiate a subject with red light and detect the red light transmitted through or reflected from the subject;
an infrared light sensor configured to irradiate the subject with infrared light and detect the infrared light transmitted through or reflected from the subject;
a control device configured to process detection signals from the red light sensor and the infrared light sensor; and
a display unit configured to display information obtained by the control device, wherein
the control device
generates a measurement data group by measuring a blood oxygen saturation of the subject based on the detection signals, removes an abnormal value from the measurement data group to obtain an oxygen saturation data group, calculates a reliability indicator for the oxygen saturation data group, and displays the oxygen saturation data group and the reliability indicator on the display unit on the same time axis, and
performs, on a range where the reliability indicator exceeds a predetermined threshold in the oxygen saturation data group, data interpolation processing for a data missing portion removed as the abnormal value from the measurement data group, displays the threshold on the display unit, and further displays the portion subjected to the data interpolation processing in a display format different from that of the oxygen saturation data group.

4. The pulse oximeter according to claim 3, wherein
the control device calculates an interval where a blood oxygen level decreases over a predetermined time or the number of times the blood oxygen level decreases, and displays a calculation result on the display unit.

5. The pulse oximeter according to claim 3, wherein
the threshold is displayed by a mark or a shape added to the display of the reliability indicator.

6. The pulse oximeter according to claim 3, wherein
a range exceeding the threshold and a range below the threshold in the measurement data group are displayed in different display formats.

7. The pulse oximeter according to claim 6, wherein
the different display formats between the range exceeding the threshold and the range below the threshold are any one or a combination of saturation, brightness, and transparency.

8. The pulse oximeter according to claim 3, wherein
different display formats between the portion subjected to the data interpolation processing and the original measurement data group are any one or a combination of saturation, brightness, and transparency.

9. A pulse oximeter comprising:
a red light sensor configured to irradiate a subject with red light and detect the red light transmitted through or reflected from the subject;
an infrared light sensor configured to irradiate the subject with infrared light and detect the infrared light transmitted through or reflected from the subject;
a control device configured to process detection signals from the red light sensor and the infrared light sensor; and
a display unit configured to display information obtained by the control device, wherein
the control device
measures a blood oxygen saturation of the subject based on the detection signals to generate a measurement data group, calculates a reliability indicator for the measurement data group, and displays the measurement data group, the reliability indicator, and a time on the display unit, and
displays a range exceeding a predetermined threshold and a range below the threshold in the measurement data group in different display formats.

10. The pulse oximeter according to claim 9, wherein
in a state where the reliability indicator falls below the threshold, any one or a combination of the following is performed: displaying a character or a symbol regarding a possibility of an abnormal value due to a body motion, displaying the reliability indicator in a display format different from that in a state where the reliability indicator exceeds the threshold, and displaying the measurement data group in a display format different from that in the state where the reliability indicator exceeds the threshold.
